(19) United States
(12) Patent Application Publication
(10) Pub. No.: US 2025/0204858 A1
(43) Pub. Date: Jun. 26, 2025

(54) MONITORING SYSTEM

(71) Applicant: Verso Biosense Limited, Abingdon, Oxfordshire (GB)

(72) Inventors: Michael POLSON, Lampeter Velfrey (GB); Matthew POWELL, Nantyglo (GB); Joseph CEFAI, Swansea (GB)

(21) Appl. No.: 18/852,072

(22) PCT Filed: Mar. 23, 2023
(86) PCT No.: PCT/GB2023/050739

§ 371 Date: Sep. 27, 2024

(30) Foreign Application Priority Data
Mar. 30, 2022 (GB) 2204608.0

(51) Int. Cl. A61B 5/00; A61B 5/07; H01F 27/28; H02J 50/10

(52) U.S. Cl. CPC A61B 5/6875; A61B 5/07; H01F 27/2823; H02J 50/10; A61B 2560/0219

(57) ABSTRACT

A first aspect of the invention provides a monitoring system, comprising: an implantable sensor device, shaped and dimensioned for implantation in a human body for measuring conditions within the human body to generate sensor data, the sensor device having a single axis antenna; and a wearable receiver device, for wirelessly receiving the sensor data generated by the implantable sensor device, wherein the receiver device comprises Helmholtz coils, wherein the Helmholtz coils are arranged to communicate with the single axis antenna so as to wirelessly charge the sensor device and receive the sensor data.

### FIELD OF THE INVENTION

The present invention relates to a monitoring system including an implantable device and a receiver device for communicating with the implantable device, and for wirelessly charging the implantable device.

### BACKGROUND OF THE INVENTION

Implantable devices often rely on wireless communication and recharging capabilities with an external device. It is challenging to perform this communication and charging, particularly when the implantable device (and therefore its antenna) is oriented in vivo, based on the body cavity in which it is implanted.

The present invention is intended to address certain of these limitations.

### SUMMARY OF THE INVENTION

A first aspect of the invention provides a monitoring system, comprising: an implantable sensor device, shaped and dimensioned for implantation in a human body for measuring conditions within the human body to generate sensor data, the sensor device having a single axis antenna; and a wearable receiver device, for wirelessly receiving the sensor data generated by the implantable sensor device, wherein the receiver device comprises Helmholtz coils, wherein the Helmholtz coils are arranged to communicate with the single axis antenna so as to wirelessly charge the sensor device and receive the sensor data.

The sensor data may be physiological data and may be one or more of pH, temperature and dissolved oxygen concentration. The sensor data additionally or alternatively be glucose concentration, lactate concentration, pressure and hormone levels.

The single axis antenna may be configured to operate at a frequency in the range 119 kHz to 140 kHz. Preferably the single axis antenna may be configured to operate at a frequency in the range 130 kHz to 140 kHz. More preferably still, the single axis antenna may be configured to operate at a frequency of 139.6 kHz. The single axis antenna may be configured to operate at a frequency of 134.2 kHz during charging of the implantable sensor device. The single axis antenna may be configured to operate at a frequency of 123.3 kHz or 134.2 kHz for transmitting or receiving data.

A first Helmholtz coil may be positioned on a first side of the single axis antenna, and a second Helmholtz coil may be positioned on a second side of the single axis antenna.

A distance between the first and second Helmholtz coils may be approximately 250 mm to 500 mm, preferably 350 mm to 450 mm, and more preferably approximately 400 mm.

The Helmholtz coils may have a radius selected to optimise both the coupling efficiency with the single axis antenna and the peak magnetic field on the axis of the coil.

The sensor device may be implantable in a human uterus.

The implantable sensor device may comprise a body and one or more arms, the arms projecting laterally from the body to secure the sensor within the uterus.

The wearable receiver device may be a wearable garment.

The wearable garment may be underwear.

A first Helmholtz coil may be positioned in a first side of the garment and a second Helmholtz coil is positioned in a second side of the garment, such that when the garment is worn by a user the first Helmholtz coil is located on an opposite side of the user to the second Helmholtz coil.

Each Helmholtz coil may comprise a principal axis, the principal axes of the Helmholtz coils configured, when the implantable device is implanted within a user and the garment is worn by the user, to substantially align with the single axis antenna of the implanted device.

The Helmholtz coils may be arranged to follow contours of the human body when the garment is worn by a user.

The receiver device may comprise two Helmholtz coils.

The receiver device may comprise four Helmholtz coils.

The Helmholtz coils may be planar wound wire.

The Helmholtz coils may be provided between two moisture sealing layers.

The Helmholtz coils may comprise copper tracks provided on polyimide film.

According to a second aspect of the invention there is provided a method of monitoring a human body, comprising: generating sensor data using an implantable sensor implanted in the human body; transmitting the sensor data via a single axis antenna to a wearable receiver device; and wirelessly charging the sensor device using Helmholtz coils of the receiver device, wherein the single axis antenna communicates with the Helmholtz coils to transmit the sensor data.

The single axis antenna may be configured to operate at a frequency in the range 119 kHz to 140 kHz, preferably 130 kHz to 140 kHz, preferably 139.6 kHz. The single axis antenna may be configured to operate at a frequency of 134.2 kHz during charging of the implantable sensor device. The single axis antenna may be configured to operate at a frequency of 123.3 kHz or 134.2 kHz for transmitting or receiving data.

### BRIEF DESCRIPTION OF THE DRAWINGS

Embodiments of the present invention will now be described by way of example only with reference to the accompanying drawings where like parts are provided with corresponding reference numerals and in which:

FIG. 1 schematically illustrates an intra-uterine monitoring system according to an embodiment of the invention;

FIG. 2 schematically illustrates an implantable sensor device according to one embodiment;

FIG. 3 schematically illustrates an implantable sensor device according to another embodiment;

FIG. 4A schematically illustrates a front view of a wearable garment according to one embodiment;

FIG. 4B schematically illustrates a back view of the wearable garment according to the embodiment of FIG. 4A;

FIG. 5 schematically illustrates the wearable garment of the embodiment of FIG. 4A in use;

FIG. 6 schematically illustrates a wearable garment according to another embodiment;

FIG. 7 is a schematic block diagram of an implantable sensor device;

FIG. 8 is a more detailed schematic block diagram of an implantable sensor device;

FIG. 9 is a schematic block diagram of a wearable receiver device;

FIG. 10 is a schematic flow diagram of the operation of the implantable sensor device;

FIG. 11 is a schematic flow diagram of a multi-charging based operation of the monitoring system;

FIG. 12 is a schematic flow diagram of a multi-antenna procedure; and

FIG. 13 is a table comparing parameters of the Helmholtz coils with those of a prior system.

### DETAILED DESCRIPTION OF EMBODIMENT(S)

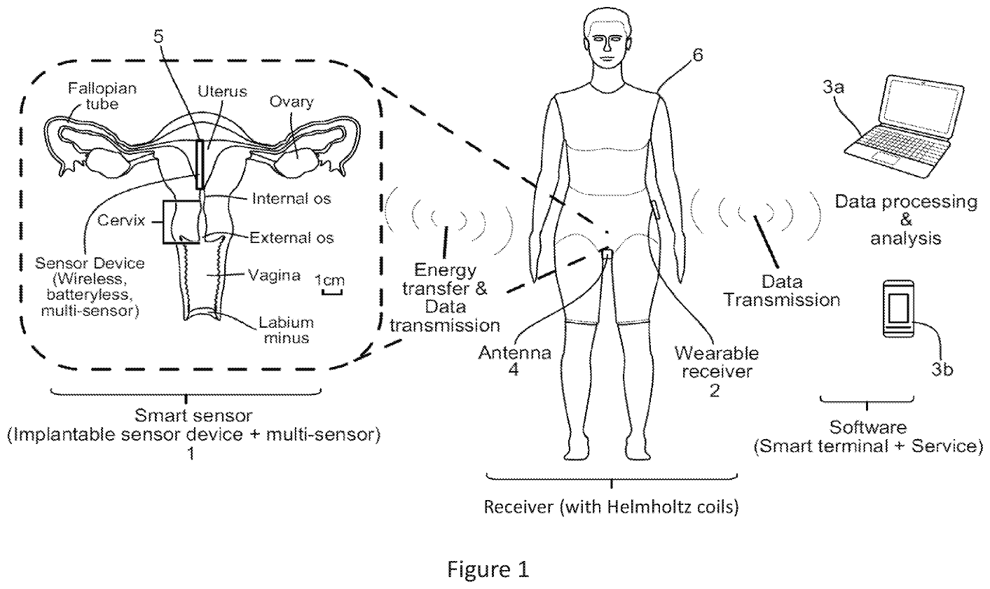

Referring to FIG. 1, a three-module structured multi-parameter in-vivo sensing platform for intra-uterine environment monitoring is shown. The platform comprises a smart sensor 1, which is suitably shaped and dimensioned for implantation in a human uterus, an external, generally wearable, receiver 2 and monitoring software installed on suitable data processing hardware, such as a computer 3a or portable electronic device 3b. The smart sensor 1 is a fully implantable (within the uterus 5 of a human female body 6) sensor device incorporating multiple sensors for monitoring physiological parameters including temperature, pressure, electrical stimuli, dissolved oxygen concentration (DOC), pH, glucose, lactate, other metabolomes, hormones, and any other physiological marker for which appropriate sensors might exist. Temperature, DOC and pH are considered to be the three most important parameters to measure in the uterus because they maintain a homeostatic controlled balance of gases and acid-base which is vital to human life and reproduction. They are likely to determine the receptivity of the intra-uterine environment to an implanting embryo.

The smart sensor 1 is capable of wirelessly receiving power from and wirelessly transmitting data to the wearable receiver 2 which is located outside the body of a user, and worn by the user. As a result, the smart sensor 1 dispenses with the need for a battery and cables, and is of comparable size to the widely-used IUDs (intra-uterine devices) for contraception. This is important, because for implantation in the uterus, a device must meet strict size limits. Compared with intra-uterine devices (IUDs) widely used for contraception, battery-based sensors have been found to be too large to be used in the uterus. Moreover, designs based on a battery typically have limitations due to the physical size of the battery and short lifetime before the battery is too depleted to continue operating. Furthermore, there are potential risks from the toxic material of battery.

The receiver 2 serves as a medium between the implantable sensor device 1 and the external data processing device running suitable software (and thus operating as a data analyser). In particular, the receiver 2 delivers energy to the sensor device and collects real-time information. An antenna 4 of the receiver 2 can be embedded into clothing and wired to the receiver 2. The software module is developed for in-vivo data uploading simultaneously to smart terminals or PC servers for post data processing and analysis. The software module consists of a set of monitoring software running on a PC or smart terminal which is designed to be a friendly user interface for data processing and system configuration. The positioning of the smart sensor 1 within the uterus is shown in FIG. 1. In particular, the smart sensor 1, which may typically have a generally elongate structure, is positioned substantially upright (vertical) within the uterus. As a result, the longitudinal axis of the smart sensor 1 is substantially vertical when the user is standing.

In this three-module structured system, the effectiveness of the wireless energy transfer and data communication between the smart sensor 1 and receiver 2 directly affect the usability of the intended system. An optimised design may not only result in better performance, smaller size, low power consumption and lower cost, but also improve end-user experience and clinical practise.

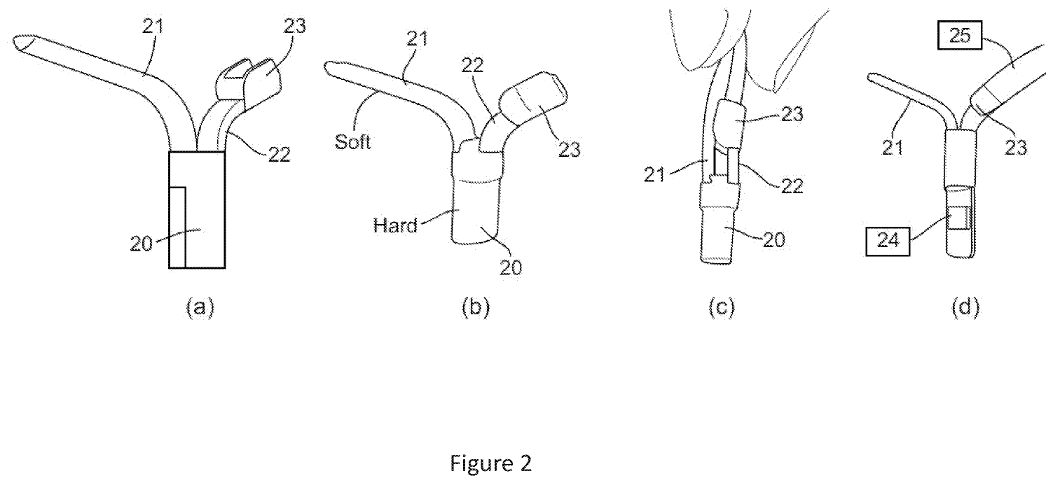

Referring to FIG. 2, an example structure for the smart sensor 1 is shown. In FIG. 2(a), an upper portion of a T-type smart sensor can be seen to comprise a middle connector 20, a first arm 21, and a second arm 22 having a connector 23. In FIG. 2(b), it is explained that the middle connector 20 is formed of a different material with a different hardness than the first arm 21 and joint of the second arm 22. In particular, a relatively hard material is used for the main body and the connector 23, whereas a relatively soft material is used to form the first arm 21 and the joint on the second arm 22. From FIG. 2(c) is it shown how the first and second arms 21, 22 are bent together during delivery into the uterus and removal to aid insertion/removal. Once inserted, the first and second arms 21, 22 help to retain the smart sense 1 in place within the uterus. While in the present embodiment two arms are used, it should be understood that in other embodiments a single arm could be used, or more than two arms could be used. In FIG. 2(d), it can be seen that a main circuit board 24 is connected to the middle connector 20 and an antenna is fixed on the second arm 22 via the connector 23. The main circuit board 24 carries the sensors and the circuitry which will be described in detail below. In this arrangement, one of the arms serves both to help keep the smart sensor 1 in place within the uterus, and also serves as the antenna for communicating with (and receiving power from) the receiver 2. In the FIG. 2 smart sensor, the antenna is generally horizontal within the uterus, making it suitable for use with a receiver having an antenna embedded in upper garments. In alternative embodiments the antenna may not form part of an arm, but may be provided elsewhere.

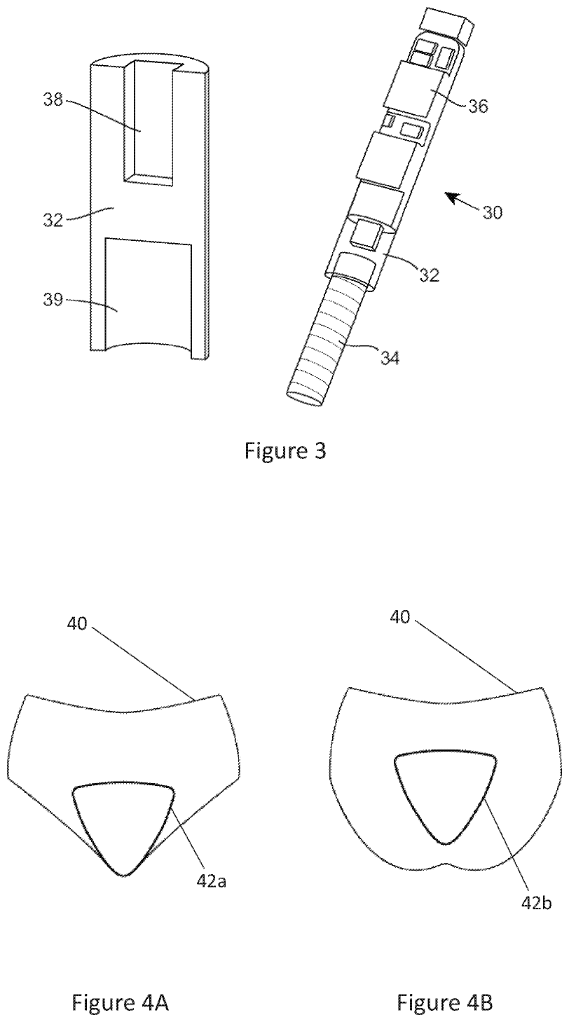

Referring to FIG. 3, a simplified structure for the smart sensor is shown. The left hand side of FIG. 3 shows a cross section of a middle connector 32 and the right hand side of FIG. 3 shows a full smart sensor 30 including the internal connection of the middle connector 32. The middle connector 32 has two sockets, present here as a rectangular slot 38 for connection to a main circuit board 36 (with sensors and circuitry on board), and a circular slot 39 for connection to a tube antenna 34. The sensor device orientation of FIG. 3 is vertical in the uterus, making it suitable for use with a receiver having a belt antenna or an antenna embedded in underwear (or a disposable sanitary towel). It will be appreciated that the sensor device of FIG. 3 could be provided with one or more arms to assist with stabilising its position within the uterus if desired. Compared with other antenna types, a tube antenna can achieve a relatively small size and tight wind on a ferrite core because it does not require a coil frame. Considering fabrication complexity and the need for the sensor to be as small as possible, a tube antenna with a ferrite core is deemed as a particularly suitable antenna for the implantable sensor device. The antenna 34 is a single axis antenna, meaning that it is a coil antenna wound onto a core so that the wire is arranged circumferentially around only one axis of the core.

FIG. 4A shows a receiver 2 in the form of a wearable garment comprising Helmholtz coils, in this case underwear 40. The front view is shown. The underwear 40 comprises a first Helmholtz coil 42a positioned on the front of the underwear 40. The first Helmholtz coil 42a is positioned so as to substantially conform with the wearer's body contours when worn, such as lying along the groove where the user's leg joins their pelvis. FIG. 4B shows the back view of the underwear 40. The underwear 40 comprises a second Helmholtz coil 42b positioned on the back of the underwear 40.

Each Helmholtz coil is a planar wound wire that is protected from moisture ingress by a moisture sealing barrier, such as by sandwiching the wire between two moisture sealing layers. Typically, the Helmholtz coils comprise copper tracks provided on polyimide film.

As shown, the Helmholtz coils 42a, 42b are substantially triangular in shape. However, the Helmholtz coils 42a, 42b may be any other suitable shape, such as circular. In this example, the first Helmholtz coil 42a is substantially the same size as the second Helmholtz coil 42b. In other examples, the first and second Helmholtz coils 42a, 42b may be different sizes. For example, the first Helmholtz coil 42a may be larger (in cross-section) than the second Helmholtz coil 42b, or vice versa. The size of the Helmholtz coils 42a, 42b may be modified to adapt to the contours of the wearer. Preferably, the first Helmholtz coil 42a arranged at the front of the underwear 40 is smaller than the Helmholtz coil 42b at the back of the underwear.

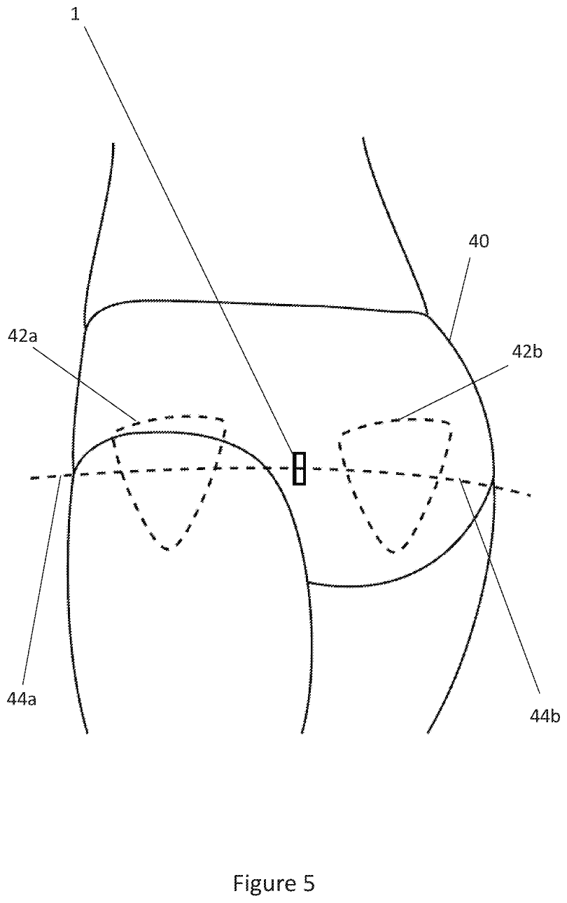

FIG. 5 shows the underwear 40 being worn by a user. The user also has a smart sensor device 1 implanted in their uterus, as described above. The smart sensor device 1 has a single axis antenna. As described previously, the underwear 40 comprises first and second Helmholtz coils 42a, 42b located on the front and back of the underwear 40 respectively. The coils 42a, 42b are positioned such that the first Helmholtz coil 42a is positioned on a first side of the single axis antenna 34 and the second Helmholtz coil 42b is positioned on a second side of the single axis antenna 34. As the first Helmholtz coil 42a is positioned in a first side of the garment and the second Helmholtz coil 42b is positioned in a second side of the garment, it can be seen that when the garment is worn by the user, the first Helmholtz coil 42a is located on an opposite side of the user to the second Helmholtz coil 42b.

Each Helmholtz coil 42a, 42b defines a respective principal axis 44a, 44b passes through the centre of the coil and runs perpendicular to the plane of the coil. As can be seen, the principal axes 44a, 44b of the two Helmholtz coils 42a, 42b substantially intersect at the location of the implanted sensor device 1. Therefore, the principal axes 44a, 44b of the two Helmholtz coils 42a, 42b and the implanted sensor 1 are substantially aligned, i.e. the principal axes 44a, 44b of the Helmholtz coils 42a, 42b and the implanted sensor device 1 are arranged to be in a substantially straight line. Therefore, when an electric current is applied to the Helmholtz coils, an extremely uniform magnetic field is generated between them, located in which is the smart sensor 1. The smart sensor 1 can therefore be wirelessly charged from this magnetic field using its single axis antenna.

The direction of the electric current in each of the coils can be applied in the same direction, clockwise or anticlockwise, through both front and back coils when viewed anteriorly to the body to generate a magnetic field that essentially aligns with a sensor single axis antenna that is located between the transverse plane of the body and close to, but not aligned to the coronal plane of the body. Alternatively, the direction of the electric current in each of the coils can be applied in a different direction through the front coil relative to the back coil to generate a magnetic field that essentially aligns with a sensor single axis antenna that is aligned to the coronal plane of the body. Thereby offering the ability to generate an appropriately-directioned magnetic field depending on the orientation of the single axis antenna. This orientation will likely depend on the orientation of the uterus in which the device is implanted, and it is typically not possible to alter this in vivo orientation. It is therefore important to be able to control the orientation of the applied magnetic field, in the manner described.

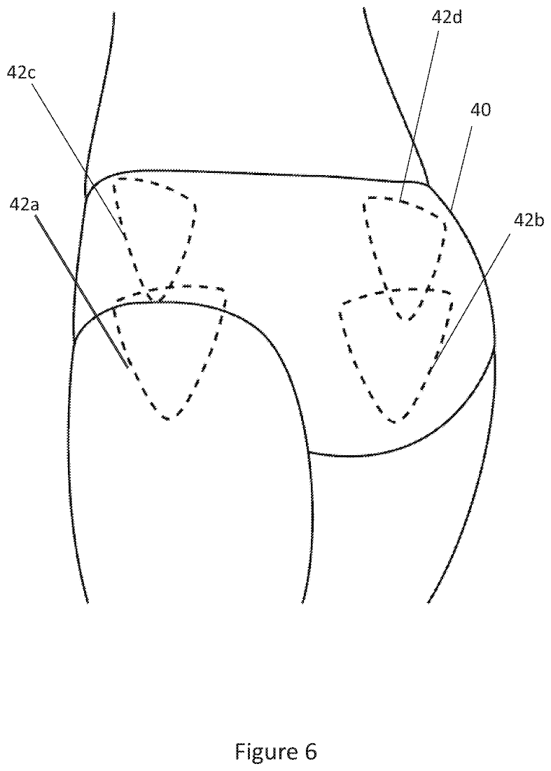

FIG. 6 shows a wearable garment in the form of underwear 40 having four Helmholtz coils 42a-d. In addition to the two coils located on the front and back of the underwear 40, there is an additional coil located on each side of the underwear 40. The four coils 42a-d are provided as two pairs, with two Helmholtz coils 42a, 42c at the front of the underwear 40 and two Helmholtz coils 42b, 42d at the rear of the underwear 40. Though not shown in this Figure, as before the Helmholtz coils each have a principal axis which substantially intersect at the location of the implanted smart sensor device 1. The Helmholtz coils 40a-d are provided as two pairs. For example, the Helmholtz coil 42a may be paired with Helmholtz coil 42b, while Helmholtz coil 42c may be paired with Helmholtz coil 42d. In this example, each pair are arranged substantially opposite each other. In other examples, the Helmholtz coil pairs 42a-d may be arranged diagonally from each other (e.g. Helmholtz coil 42c may be connected to Helmholtz coil 42b, while Helmholtz coil 42a may be connected to Helmholtz coil 42d). Using four coils instead of two has the benefits of providing greater control over the orientation of the magnetic field.

A similar embodiment is a wearable garment in the form of underwear 40 having four Helmholtz coils. In this embodiment two coils are located on the front of the garment and arranged so that one coil is positioned above the other coil when worn. Two coils are located on the back of the garment and arranged so that one coil is positioned above the other coil when worn. By alternating which coil at the front of the garment and which coil at the back of the garment electric current is supplied to, a greater range of magnetic field locations and alignments with the implant can be realised.

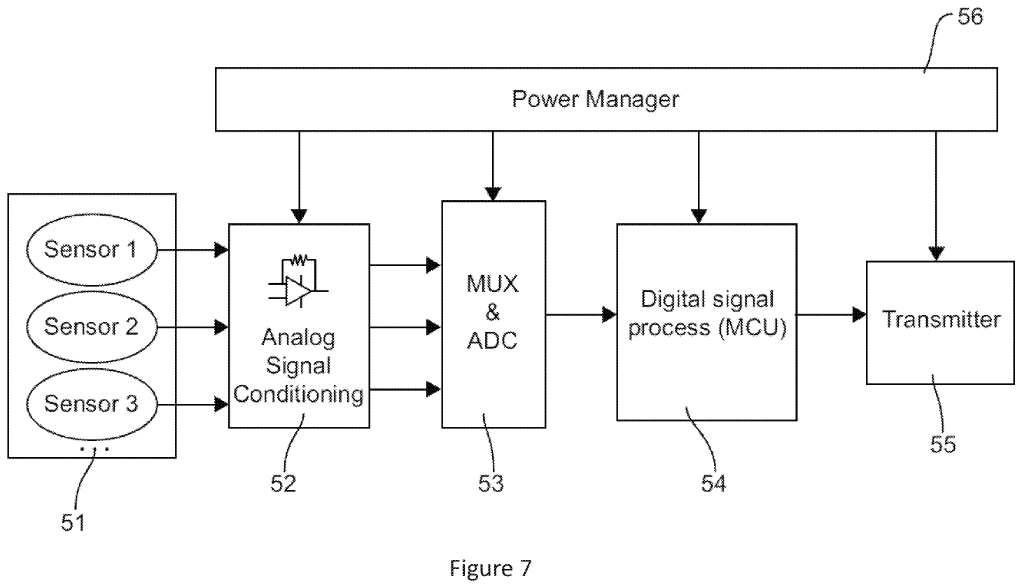

Referring to FIG. 7, a block diagram of a suitable microsystem structure for the smart sensor is shown. To achieve data capture, signal processing and wireless communication, the microsystem includes integrated sensors or a multi-sensor array 51, an analogue signal conditioning circuit 52, an analogue to digital converter (ADC) 53 optionally including a multiplexer (MUX), a digital signal processor 54 or micro-programmed control unit (MCU), a wireless transmitter 55 and a power manager 56. In operation, the sensors 51 convert physical parameters into electronic signals. Sensors can be fabricated on tiny silicon chips based on micro-fabrication technologies which are suitable for microsystems. The conditioning circuit 52 is used to improve the quality of the analogue signals from the sensors 51. Generally, high end conditioning performance requires more complex circuits. A simpler conditioning circuit results in limited performance that requires further data processing after analogue-digital conversion. Therefore, a balance between signal conditioning performance and circuit complexity should be considered for the system implementation. A multiplexer (MUX) may be employed for circuit hardware sharing, which has the advantage of reducing device size and power consumption. The processer 54 undertakes logic control and digital signal processing. A micro-programmed control unit (MCU) is a widely-used component for flexible functionality and good scalability. At the same time, compared with a powerful processor, an MCU enables power consumption to be reduced. The wireless transmitter 55 and power manager 56 are provided for data transmission and power control respectively. Integration of these different units can facilitate device miniaturisation.

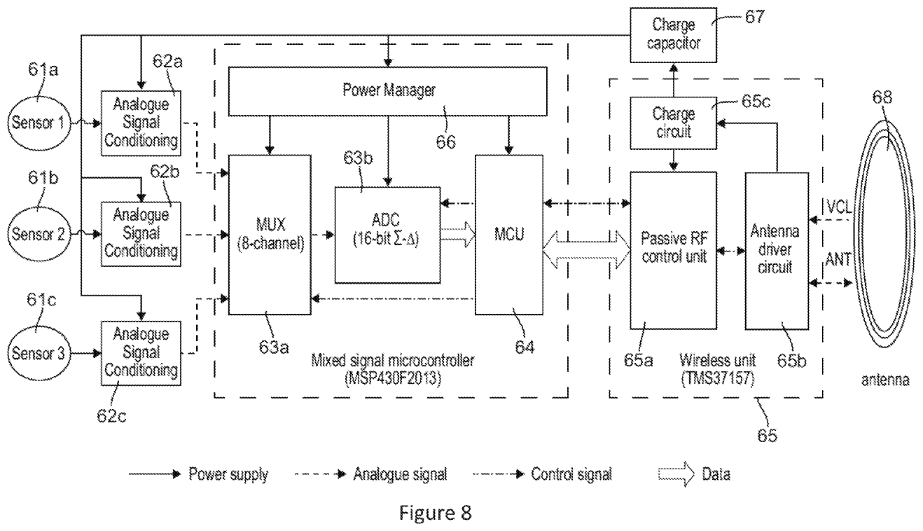

Referring to FIG. 8, the main structure of the sensor device is shown in greater detail, including sensors 61a, 61b, 61c (temperature, DOC and pH in this case), respective analogue signal conditioning units 62a, 62b, 62c (one for conditioning the output of each of the sensors 61a, 61b, 61c), a multiplexer 63a (to allow an array of sensors to be interfaced with the ADC), analogue to digital converter 63b and MCU 64, and a transmitter 65. Two-way communication with the MCU from outside the body is also possible, for example to wake it from a power saving sleep-state. Furthermore, the antenna can be used for wireless power transfer through inductive coupling. The transmitter 65 here comprises a passive RF control unit 65a for communicating with the MCU 64, and controlling an antenna driver circuit 65b and a charging circuit 65c. The antenna driver circuit is capable of bidirectional data communication with the external receiver (Antenna, ANT) via an antenna (in this case a Helmholtz coil antenna) 68, as well as receiving power (charging voltage, VCL) via the antenna 68. It will therefore be understood that the system utilises wireless power transfer through inductive coupling, using a low frequency RFID signal to transmit data and receive power through the abdominal region of a user. The charge circuit 65c is able to charge a capacitor 67 and also deliver power to the control unit 65a using the electrical power received via the antenna 68 and driver circuit 65b. All power is managed through the power management block 66. The flow of power around the circuitry of FIG. 8 is indicated by solid directional arrows. Analogue data signal flow is indicted by a first type of dashed directional arrow. Control signal flow is indicated by a second type of dashed directional arrow. Data flow is indicated by solid block arrows. It can be seen that the charge capacitor 67 delivers electrical power to the power manager 66 (which in turn manages power delivery to the multiplexer 63a, the ADC 63b and the MCU 64), as well as to the signal conditioning units 62a, 62b, 62c. The capacitor 67 is preferably a ceramic capacitor. This type of capacitor is particularly suitable for the present purposes for safety reasons, for example because it does not utilise toxic materials and has little or no risk of leakage.

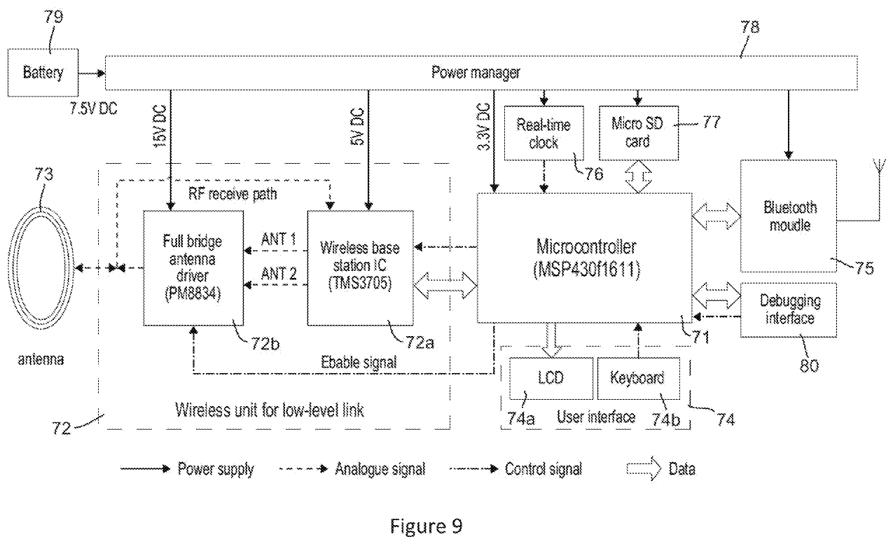

Referring to FIG. 9, an overview of the receiver structure is shown. A microcontroller 71 links and controls different peripherals. A low-level wireless unit 72 provides wireless energy transfer to and data communication with the implantable sensor device via a Helmholtz coil antenna 73. The wireless unit 72 comprises a wireless base station integrated circuit 72a for implementing an RF analogue front end to generate the antenna driving signal, and to modulate and demodulate the digital signal, and a full bridge antenna driver 72b for providing the output power and driving the antenna. A user interface 74 comprising an LCD display 74a and a keyboard 74b is able to display received sensor data and system information provided by the microcontroller 71, and to offer a facility for a user to operate the device, again via the microcontroller 71. A Bluetooth module 75 provides high-level communication with servers or smart terminals, where data analysis can be performed. A real-time clock 76 provides time information, for example to give a time stamp to each data item, and to achieve continuous measurement, and a micro SD card interface 77 provides local data storage. A power manager 78 converts a power supply from a rechargeable battery 79 to match the different voltage requirement of the peripherals. The flow of power around the circuitry of FIG. 6 is indicated by solid directional arrows. Analogue data signal flow is indicted by a first type of dashed directional arrow. Control signal flow is indicated by a second type of dashed directional arrow. Data flow is indicated by solid block arrows.

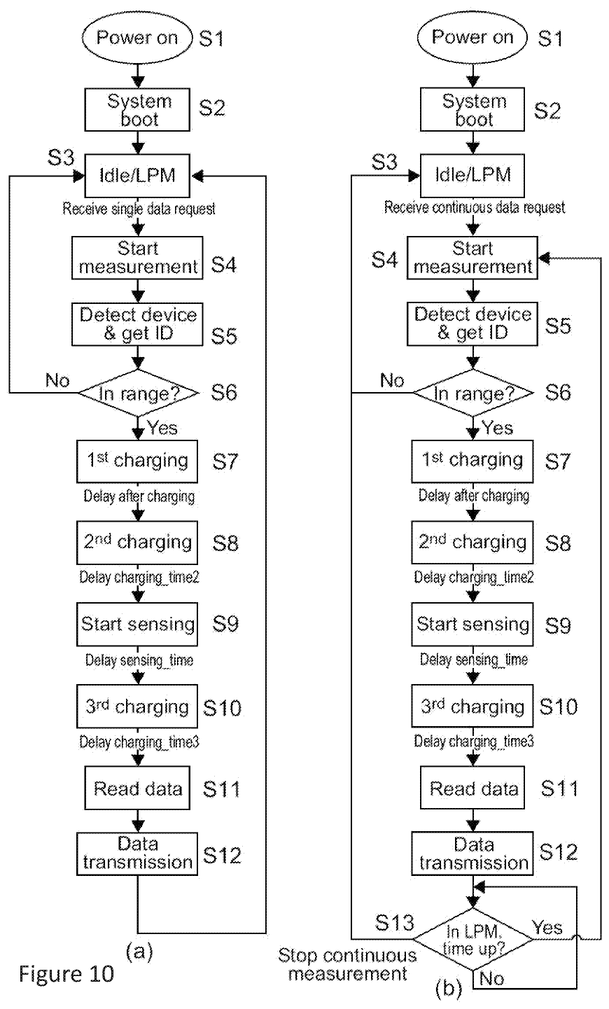

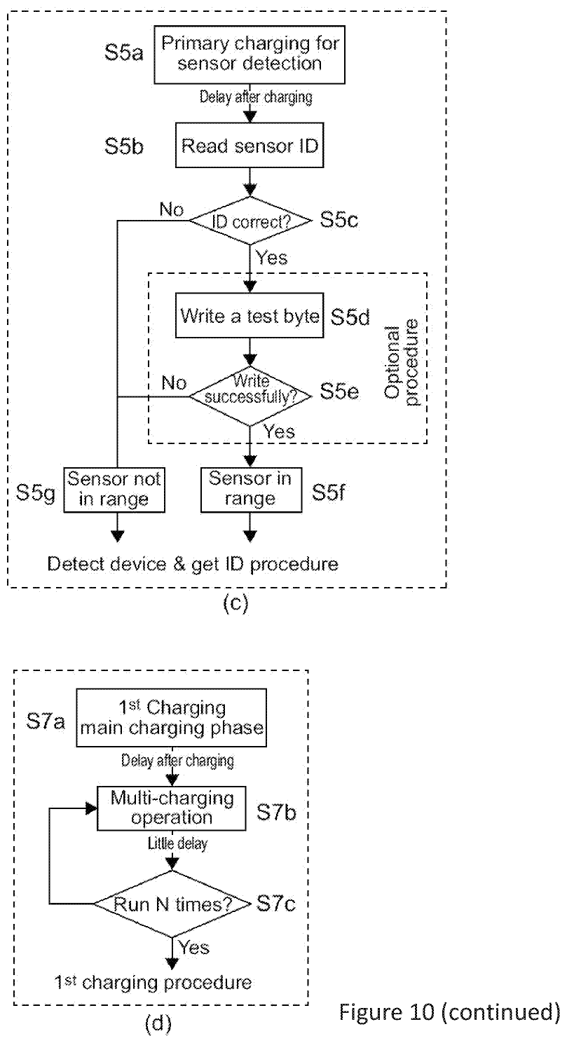

Referring to FIG. 10, flow charts of operating procedures for single sampling (a) and continuous sampling (b) are shown. For single sampling, at a step S1 the system is powered on, and then boots at a step S2, initialising the various circuitry shown in FIG. 9. After this, the system enters an idle or lower power mode at a step S3, which it remains in until it receives a single data request (for example in response to a user actuating the wearable receiver or a wirelessly connected control device. In response to such a trigger, at a step S4 a sampling/measurement cycle is initiated. At a step S5, the receiver attempts to detect the smart sensor device and obtain its identifier. This procedure is described in more detail in FIG. 10(c). If at a step S6 it is determined that the sensor device is not in range and cannot be detected, then the process returns to the idle/lower power state S3. If at the step S6 the sensor device is found to be in range, then at a step S7 a first charging operation is conducted. The first charging operation is described in more detail in FIG. 10(d). The first charging operation is intended to provide the sensor device with sufficient power to carry out a boot procedure in which the circuitry of FIG. 8 is powered up and initialised. More specifically, the booting procedure may include hardware initialisation, MCU state initialisation, working parameter initialisation (firmware) and entering the low-power mode immediately after the booting, pending instructions to start operation. It will be appreciated that the boot procedure will use some or all of the power provided in the first charging operation. The step S7 is followed, after a short delay (during which the sensor device will be booting up) by a second charging operation at a step S8, which replenishes the charge in the capacitor of the sensor device, whereupon the sensor device is able to start sensing (but not transmitting) at a step S9. At a step S10, a third charging operation is used to transfer enough energy to the sensor device for data to be read from the sensors at a step S11, and for wireless data transmission of the sensor data to take place at a step S12. After data transmission, the system returns to the idle mode at the step S3 and waits for a next sampling cycle. The procedure for continuous sampling (b) is similar to a single sampling cycle, and corresponding steps are labelled using the same step numbers. The difference here is that the sampling cycle runs automatically at pre-set time intervals, governed by a step S13, which is able to either keep the system idle or in the low power mode, cancel the task or trigger the start of the operation by returning the process to the step S4. In effect, after data transmission at the step S12, the system enters a sleep mode and a timer counts down to trigger the next sampling cycle. If a “Cancel” button is triggered, the system would return back to the idle mode. This procedure serves to transfer energy efficiently, limit crosstalk noise between energy transfer and data transmission due to the shared link and shorten the sampling cycle. Multi-step charging is adopted for the sensor device rather than single charging, as it ensures sufficient energy for each working step and less overall charging time than single charging.

Referring to FIG. 10(c), the detect device and obtain ID procedure (step S5) is described in greater detail. In particular, at a step S5a, a primary charging operation for sensor device detection is carried out. This need only charge part of the sensor device, for example the wireless unit 65, which is sufficient for the sensor ID to be read at a step S5b, a short time after the charging step S5a has taken place. At a step S5c it is determined whether the read ID is correct. If not (for example the ID is unrecognised, or corrupted due to a poor wireless connection), then at a step S5g a signal is generated (for use by the step S6) indicating that the sensor device is not in range. If however the ID is determined to have been read correctly at the step S5c, then a test byte is written to the sensor device at a step S5d. It is then determined at a step S5e whether the writing of the test byte has been successful. If not, then again, a not in range signal is generated at the step S5g. If however the test byte is determined to have been written successfully, then at a step S5f a signal is generated (for use by the step S6) indicating that the sensor device is in range. The test write procedure of the step s5d and S5e is an optional procedure for confirming the reliability of the wireless connection between the sensor device and the receiver.

Referring to 10(d), the first charging procedure of the step S7 is described in more detail. In particular, at a step Sa a first charging main charging phase is carried out. This is a single, continuous charging phase in which power is delivered to the sensor device to charge the capacitor. The main charging phase may for example charge the capacitor to 50% of capacity relatively quickly. However, continuous charging beyond this may cause excess heating of the sensor device or components thereof. Accordingly, the step S7a is followed by multiple iterations of steps S7b (which transfers a small amount of power to the sensor device) and S7c (which cycles back to the step S7b until it has been carried out N times). The combinations of the steps S7a, S7b and S7c fully charge the capacitor. The use of multiple short charges controls the heating effect by limiting the continuous generation of heat at the coil and power driver circuit (on the wearable receiver), and for the implantable sensor inhibits overheating on the coil.

The low power state may be a “sleep” state in which the device is substantially powered down to conserve power, but is capable of being woken up to operate. In contrast, the idle state may be an operational state in which the device is merely awaiting an instruction. The device may be quicker to react when in the idle state than in the lower power (sleep) state.

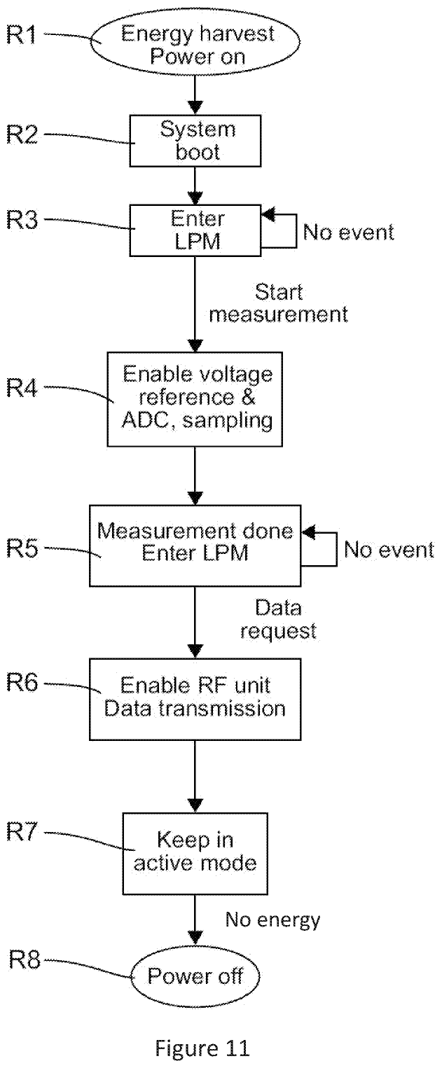

Referring to FIG. 11, a flow chart of the operation of the implantable sensor device is shown. FIG. 11 sets out the operation of the sensor device following its detection at the steps S5 and S6 of FIG. 10. At a step R1, energy is harvested at the sensor device by virtue of the step S7 (first charging procedure) and is used to power on the sensor device and perform a system boot at a step R2, as discussed above. At a step R3, the sensor device enters a low power mode, which continues while no events occur. If the sensor device receives an instruction to start measurement from the receiver device, then at a step R4 the sensor device enables a voltage reference and the analogue to digital converter circuitry, and start taking measurements using the sensors. The step R4 is carried out using power provided to the sensor device at the second charging procedure at S8 of FIG. 10. Once the measurements have been made, and stored locally, the sensor device again enters the low power mode at a step R5, which again persists until interrupted by an instruction from the receiver device (data request), at which point the process moves on to a step R6, where the RF unit of the sensor device is enabled, and transmission of sensor data from the sensor device to the receiver device takes place. The step R6 is carried out using power provided to the sensor device at the third charging procedure at the step S10 of FIG. 10. Following the step R6, the sensor device then remains in the active mode at a step R7 until the capacitor is exhausted, at which point the sensor device powers off at a step R8. The reason for actively exhausting the capacitor is so that the sensor device is fully reinitialised each time it is used, and so that the capacitor can be energised consistently from its empty state to a full state using the charging steps indicated in FIG. 10.

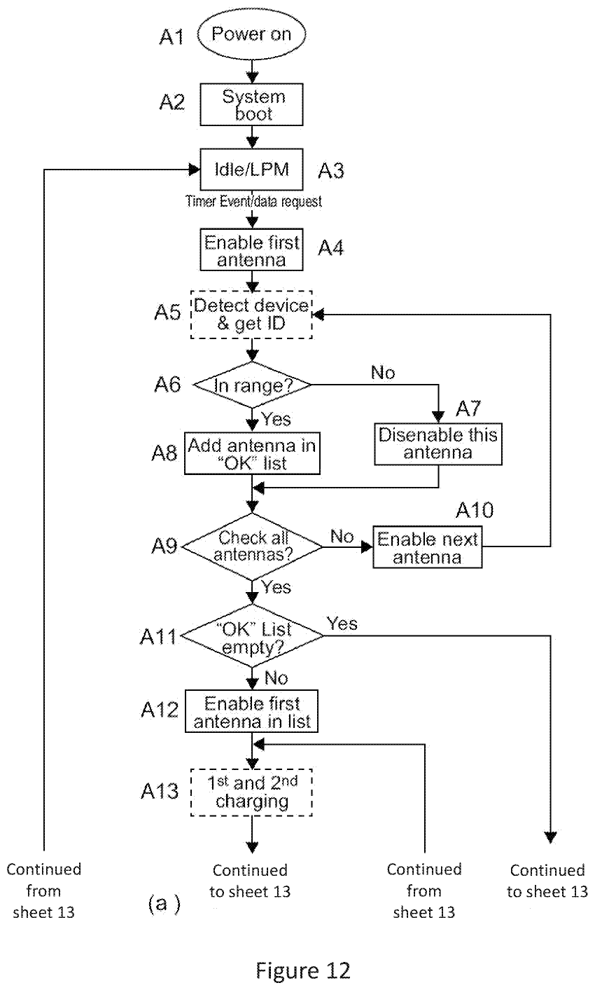

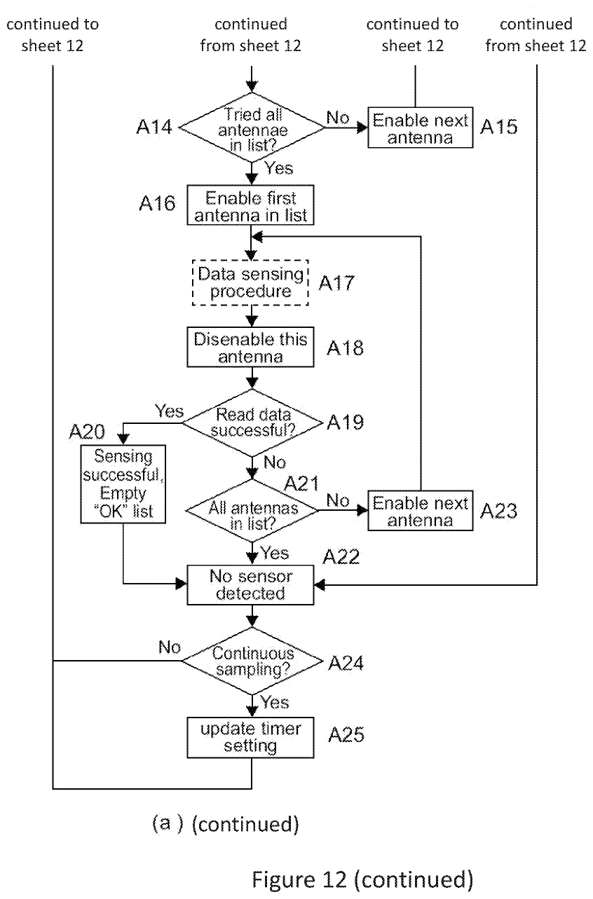

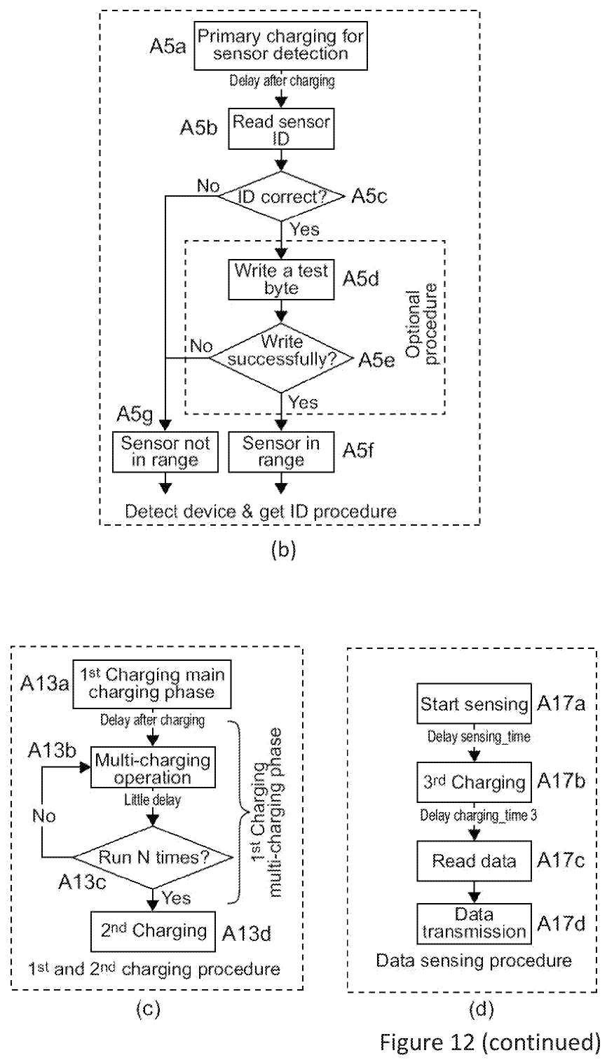

Referring to FIG. 12, flow charts of operating procedures for a multi-antenna receiver system are shown. The multiple antennae could be present in a single device, such as a wearable receiver, or a receiver utilising an antenna embedded within an item of furniture, or alternatively one (or more) antennae could be provided in a wearable form, while another one (or more) could be embedded in an item of furniture. As will be understood from the following description, the plural antennae work cooperatively to improve overall performance. Compared to a single receiver system, a challenge for a multi-antenna receiver system is how to coordinate the different antennas and perform sampling quickly and efficiently, irrespective of the position and orientation of the sensor device with respect to the various antennae. In FIG. 12(a), a working procedure for a multi-antenna receiver system is explained. At a step A1, the system is powered up. At a step A2, the system is booted in the same manner as the step S2 of FIG. 10. The system then enters an idle mode or low power mode at a step A3 and waits for an event trigger, such as a button being pressed, or a timer event in the case of continuous sampling. Once triggered, a sampling (measurement) cycle is started. To achieve this in a multi-array setup, at a step A4, a first antenna is enabled, and at a step A5 an attempt is made to detect the sensor device and obtain its identifier. This process is set out in FIG. 12(b), which corresponds precisely to FIG. 10(b). In particular, the steps A5a to A5g of FIG. 12(b) are the same as the steps S5a to S5g, and will not be described again. If it is determined at a step A6 that the sensor device is not in range of that antenna, then at a step A7 the first antenna is disabled. If it is determined at the step A6 that the sensor device is in range of the first antenna, then this antenna is added to an “OK” list (a list of available antennas) at a step A8. Then, at a step A9 it is determined if all antennas have been checked. If not, then at a step A10 the next antenna is enabled, and the process returns to the step A5, where the steps A5 to A8 will be repeated for the next antenna. This process continues until all antennae have been considered and either added to the “OK” list or disabled. After cycling through all antennas, at a step A11 it is determined if the “OK” list is empty. If so, then it is determined at a step A21 that no device is detected. Otherwise, the process moves onto a step A12, where the first antenna in the “OK” list is enabled. In particular, at a step A13 first and second charging procedures are carried out by the first antenna (as will be discussed in FIG. 12 (c)). At a step A14 it is determined if all antennae in the list have carried out the charge procedures. If not, then at a step A15 the next antenna in the list is enables and the steps A13 and A14 are repeated. When at the step A14 it is determined that all antennae in the list have carried out the charge procedures, then at a step A16 the first antenna in the “OK” list is selected, and triggers the data sensing procedure discussed above in relation to FIG. 10 at a step A17 (see also FIG. 12 (d)). This antenna is then disabled at a step A18. It is then determined at a step A19 whether the reading of the sensor data has been successful. If so, then it is not necessary to utilise any of the other antennae in the “OK” list, and the list can be emptied at a step A20. Otherwise, if the reading of the sensor data has not been successful then at a step A21 it is determined if all antennae have attempted to obtain sensor data from the sensor device. If so, then the process progresses to the step A22 where it is determined that no device is detected (or at least that no device can be read). Otherwise, at a step A22 the next antennae device in the “OK” list is selected, and the steps A17 to A22 are repeated for the next antennae device. Following the step A22, it is determined at a step A23 whether the continuous sampling mode is active. If not, then the process returns to the idle state at the step A3. If the continuous sampling mode is active, then a timer is set at a step A24 and the process then returns to the idle mode or low power mode at the step A3. Following the expiry of the timer, the step A4 will commence. In this way, it will be appreciated that the charging phase of all available antennae entered in the “OK” list is carried out. The charging by all available antennas (one by one) enables sufficient energy to be delivered to the device, even when the position and orientation of each individual antenna of the receiver were not optimal. Subsequently, the available antennas read the data successively after the charging phase. Once the data has been retrieved successfully, this sampling cycle is finished. If all available antennas do not get any correct data, then a failed message may be sent back to the software.

Turning to FIG. 12(c) a multi-charge procedure is shown to comprise a first charging step A13a, followed by a multi-charging operation executed N times represented by the steps A13b and A13c, followed by a second charging step A13d. Turning to FIG. 12(d), a data sensing procedure is shown in which the sensor device is instructed to start sensing at a step A17a, and then the sensor device is charged (by the receiver device) at a step A17b in order to provide sufficient power for a step A17c of reading the sensor data and a step A17d of transmitting the read sensor data to the receiver device.

As discussed above, electromagnetic induction wireless transmission technology is used for near field energy transfer through the use of two coupled coils, primary and secondary coils, provided at the receiver device and implantable sensor respectively. The electric current flowing through the primary coil creates a magnetic field that acts on the secondary coil producing an induced current within it. Tight coupling is needed for high energy harvest efficiency and long working distance. Increasing the distance between the coils results in the magnetic field extending beyond the secondary coil receiving area and leads to a loss of transmitted energy. Within the intended application, an implantable sensor device requires small size, low power consumption and a relatively short working distance of around 10 cms. Energy loss due to tissue absorption of the wireless signals (the electromagnetic energy is transformed to other forms of energy by matter within the medium of tissues, for example, to heat) is dependent on the signal frequency. Signals at lower frequencies have better propagation characteristics and result in less tissue absorption. Therefore, wireless energy transfer based on electromagnetic induction at low frequency is employed for the implantable sensor device. The circuit for wireless energy transfer can also serve as the wireless data communication as a low frequency RFID link, reducing the need for additional circuits or board space for data communication. In the intended application, the in-vivo information and system configuration do not require high data rate transmission and the LF RFID link can provide sufficient data bandwidth to meet the demand. The data communication range is usually further than the energy transfer distance meaning it is not the bottleneck of the effective working distance.

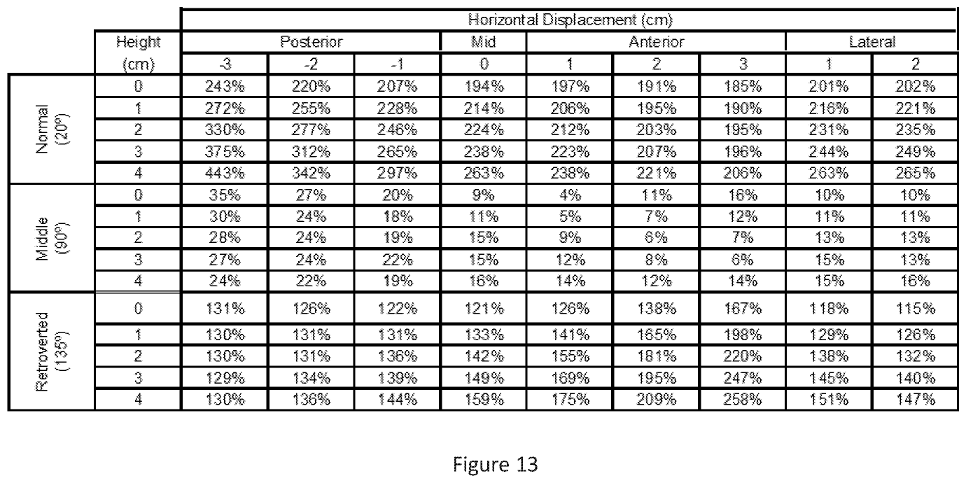

FIG. 13 shows a table illustrating the percentage improvement in induced voltage of the Helmholtz coils compared to prior technology used for wirelessly charging an implanted sensor. The table displays data relating to normal, middle and retroverted cavities within the body, as well as the positioning of the implant within the cavity itself.

While the various techniques, and the implantable sensor device and external receiver have been explained in the context of intra-uterine monitoring, it will be understood that these techniques and structures could be applied to other body-cavity monitoring, such as within a vagina, bladder or digestive tract of a human or animal body.

Although the invention has been described above with reference to one or more preferred embodiments, it will be appreciated that various changes or modifications may be made without departing from the scope of the invention as defined in the appended claims.

1. A monitoring system, comprising:
an implantable sensor device, shaped and dimensioned for implantation in a human body for measuring conditions within the human body to generate sensor data, the sensor device having a single axis antenna; and
a wearable receiver device, for wirelessly receiving the sensor data generated by the implantable sensor device,
wherein the receiver device comprises Helmholtz coils, wherein the Helmholtz coils are arranged to communicate with the single axis antenna so as to wirelessly charge the sensor device and receive the sensor data.

2. The monitoring system according to claim 1, wherein the sensor data is one or more of pH, temperature and dissolved oxygen concentration.

3. The monitoring system according to claim 1, wherein the single axis antenna is configured to operate at a frequency in the range 119 kHz to 140 kHz, preferably 130 kHz to 140 kHz, preferably 139.6 kHz.

4. The monitoring system according to claim 1, wherein a first Helmholtz coil is positioned on a first side of the single axis antenna, and a second Helmholtz coil is positioned on a second side of the single axis antenna; and optionally
wherein a distance between the first and second Helmholtz coils is approximately 250 mm to 500 mm, preferably 350 mm to 450 mm, preferably approximately 400 mm.

5. The monitoring system according to claim 1, wherein the Helmholtz coils have a radius selected to optimise both the coupling efficiency with the single axis antenna and the peak magnetic field on the axis of the coil.

6. The monitoring system according to claim 1, wherein the sensor device is implantable in a human uterus; and optionally
wherein the implantable sensor device comprises a body and one or more arms, the arms projecting laterally from the body to secure the sensor within the uterus.

7. The monitoring system according to claim 1, wherein the wearable receiver device is a wearable garment; and optionally
wherein the wearable garment is underwear.

8. The monitoring system according to claim 7, wherein a first Helmholtz coil is positioned in a first side of the garment and a second Helmholtz coil is positioned in a second side of the garment, such that when the garment is worn by a user the first Helmholtz coil is located on an opposite side of the user to the second Helmholtz coil.

9. The monitoring system according to claim 7, wherein each Helmholtz coil comprises a principal axis, the principal axes of the Helmholtz coils configured, when the implantable device is implanted within a user and the garment is worn by the user, to substantially align with the single axis antenna of the implanted device.

10. The monitoring system according to claim 7, wherein the Helmholtz coils are arranged to follow contours of the human body when the garment is worn by a user.

11. The monitoring system according to claim 1, wherein the receiver device comprises two Helmholtz coils.

12. The monitoring system according to claim 11, wherein a first electric current is driven in a first direction around a first of the Helmholtz coils when viewed from a first perspective, and a second electric current is driven in the first direction around the second of the Helmholtz coils when viewed from the first perspective.

13. The monitoring system according to claim 11, wherein a first electric current is driven in a first direction around a first of the Helmholtz coils when viewed from a first perspective, and a second electric current is driven in a second direction, different from the first direction, around the second of the Helmholtz coils when viewed from the first perspective.

14. The monitoring system according to claim 11, wherein the receiver device comprises four Helmholtz coils.

15. The monitoring system according to claim 14, wherein the four Helmholtz coils are provided as two pairs of coils, each coil of a pair of coils arranged to be positioned on an opposite side of the implantable sensor device to the other coil of the pair of coils.

16. The monitoring system according to claim 1, wherein the Helmholtz coils are planar wound wire.

17. The monitoring system according to claim 16, wherein the Helmholtz coils are provided between two moisture sealing layers.

18. The monitoring system according to claim 1, wherein the Helmholtz coils comprise copper tracks provided on polyimide film.

19. A method of monitoring a human body, comprising:
generating sensor data using an implantable sensor implanted in the human body; transmitting the sensor data via a single axis antenna to a wearable receiver device; and
wirelessly charging the sensor device using Helmholtz coils of the receiver device, wherein the single axis antenna communicates with the Helmholtz coils to transmit the sensor data.

20. The method according to claim 19, wherein the single axis antenna is configured to operate at a frequency in the range 119 kHz to 140 kHz, preferably 130 kHz to 140 kHz, preferably 139.6 kHz.
